(19) United States
(12) Patent Application Publication
(10) Pub. No.: US 2023/0405170 A1
(43) Pub. Date: Dec. 21, 2023

(54) TABLETOP STEAM STERILIZER WITH INTEGRATED WATER DISTILLER

(71) Applicant: Lucas Lifecare, LLC, Pompano Beach, FL (US)

(72) Inventors: Joseph Paul Vincent, Bangalore (IN); Randell Quaal, Pompano Beach, FL (US)

(21) Appl. No.: 18/210,785

(22) Filed: Jun. 16, 2023

Related U.S. Application Data
(60) Provisional application No. 63/352,749, filed on Jun. 16, 2022.
(60) Provisional application No. 63/352,745, filed on Jun. 16, 2022.

(51) Int. Cl. A61L 2/24; A61L 2/07; B01D 3/14; B01D 3/42; B01D 5/00

(52) U.S. Cl. CPC A61L 2/24; A61L 2/07; B01D 3/14; B01D 3/42; B01D 5/006; A61L 2202/11; A61L 2202/122; A61L 2202/14

(57) ABSTRACT

A tabletop steam sterilizer system is housed within a tabletop cabinet housing having a water inlet that is adapted for connection to a municipal water supply, thus allowing its use anywhere that there is a standard water supply and without need of a distilled water supply. The tabletop steam sterilizer system includes at least one sterilizing chamber, although more than one independently operable sterilizing chamber may be provided. The tabletop steam sterilizer system also includes a water distiller system that receives water from the municipal or other non-distilled water supply, converts that municipal or other non-distilled water to distilled water, and supplies that distilled water to the sterilization chamber to carry out a sterilization cycle. Exhaust from the sterilization chamber may be recycled back to the distiller system for processing by the distiller system back into distilled water for subsequent sterilization cycles.

### CROSS-REFERENCE TO RELATED APPLICATIONS

This application claims the benefit of U.S. Provisional Patent Application No. 63/352,749 titled “TABLETOP STEAM STERILIZER WITH INTEGRATED WATER DISTILLER,” filed by the Applicant herein on Jun. 16, 2022, and of U.S. Provisional Patent Application No. 63/352,745 titled “TABLETOP DUAL-CHAMBER STEAM STERILIZER WITH INTEGRATED WATER DISTILLER,” filed by the Applicant herein on Jun. 16, 2022, the specifications of which are incorporated herein by reference in their entireties.

### FIELD OF THE INVENTION

The present invention relates generally to medical and laboratory steam sterilizers, and more particularly to a tabletop steam sterilizer equipped with an integrated water distiller such that the sterilizer may be operated from a municipal or similar non-distilled water source, and more particularly independent of an external supply of distilled water.

### BACKGROUND OF THE INVENTION

Steam sterilizers are used to sterilize physical items such as medical devices and instruments (i.e., a “load”) in a sealed sterilizing chamber by destroying all forms of microbial life on those instruments using saturated steam under pressure. The saturated steam is generated in a steam generator that is in fluid communication with the sterilizing chamber. In the steam generator, electric resistance heating raises the temperature of water to its boiling point, in turn generating saturated steam by evaporation. To avoid scale formation, only distilled water is used in the steam generator, having total dissolved solids (“TDS”) under 10 ppm (corresponding to electrical conductivity of under 15 μS/cm at 20° C.).

Compact steam sterilizers may be preferable in certain medical and laboratory settings where space is limited. Such tabletop steam sterilizer systems typically do not have any onboard system that enables distilled water generation and delivery. Rather, users must obtain and maintain a supply of distilled water for use in each sterilizing cycle of the steam sterilizer. As the proportion of water drained out after every sterilizing program cycle is significant, steam sterilizers have in some instances been configured to reuse the residual water exhausted from the sterilizing chamber by filtering through nano-sediment filters. Unfortunately, however, such nano-sediment filters must be replaced at frequent intervals. Thus, there remains a need in the art for steam sterilizers that are capable of operating without requiring the external supply of distilled water, and that instead may generate such distilled water in the system itself.

Further, such previously known steam sterilizers typically operate in only one of the standard sterilizing cycles; namely, B, S, or N, despite the need for different kinds of loads requiring the operation of different sterilizing cycles. Thus, there also remains a need in the art for such systems to operate in all three sterilizing cycles on-demand as opposed to the limited sterilizing cycles offered by previously known sterilizing systems.

### SUMMARY OF THE INVENTION

In accordance with certain aspects of the invention, a tabletop steam sterilizer system is provided that avoids one or more disadvantages of previously known sterilizing systems. In an exemplary configuration, a tabletop steam sterilizer system is provided in a tabletop cabinet housing having a water inlet that is adapted for connection to a municipal water supply or similarly configured source of non-distilled water, thus allowing its use anywhere that there is a standard water supply and without need of a distilled water supply. The tabletop steam sterilizer system includes at least one sterilizing chamber, although more than one independently operable sterilizing chamber may be provided. The tabletop steam sterilizer system also includes a water distiller system that receives water from the municipal or other non-distilled water supply, converts that municipal or other non-distilled water to distilled water, and supplies that distilled water to the sterilization chamber to carry out a sterilization cycle. Preferably, exhaust from the sterilization chamber is recycled back to the distiller system for processing by the distiller system back into distilled water for subsequent sterilization cycles.

In accordance with certain aspects of an exemplary embodiment, a sterilizer system is provided, comprising: a housing; at least one sterilizing chamber in the housing; and a water distiller system in the housing and in fluid communication with the at least one sterilizing chamber, wherein the water distiller system has a municipal water supply inlet configured to receive water from a municipal water supply, and a distilled water outlet in fluid communication with the at least one sterilizing chamber; wherein the water distiller system is configured to convert municipal water into distilled water for supply to the at least one sterilizing chamber.

The sterilizer system may further comprise a sterilizer condenser in the housing having an inlet in fluid communication with an exhaust from the at least one sterilizing chamber and an outlet in fluid communication with the water distiller system.

The water distiller system may further comprise: a cooling tank in fluid communication with the municipal water supply inlet; a distiller system condenser; a coolant water recirculation fluid circuit between the coolant tank and the distiller system condenser and configured to recirculate water between the coolant tank and the distiller system condenser; and a supply water flow circuit from the coolant tank to an evaporating tank and from the evaporating tank back through the coolant tank and to a distilled water feeding tank, wherein the distilled water feeding tank is in fluid communication with the distilled water outlet.

The sterilizer system may further comprise a closed steam conduit extending through the coolant tank, wherein the closed steam conduit is in fluid communication with a steam outlet on the evaporating tank and a distilled water inlet on the distilled water feeding tank. The closed steam conduit may further comprise a coil positioned in the coolant tank and configured for heat transfer between steam inside of the coil and municipal water inside of the coolant tank.

The water distiller system may further comprise a recirculating tank in fluid communication with an exhaust outlet of the at least one sterilizing chamber. The sterilizer system may further comprise a sterilizer condenser receiving exhaust from the exhaust outlet and configured to convert the exhaust to water, and a recirculating tank fluid supply line between the sterilizer condenser and the recirculating tank. The recirculating tank may further comprise a recirculating tank outlet in fluid communication with an evaporating tank inlet on the evaporating tank.

The sterilizer system may further comprise a controller configured to initiate evaporating cycles in the evaporating tank based on a detected volume of distilled water in the distilled water feed tank. The controller may be further configured to cause the sterilizer system to automatically flush residual dissolved solids after each evaporating cycle.

In accordance with further aspects of an exemplary embodiment, a sterilizer system is provided, comprising: a housing; a sterilizing chamber in the housing; a distilled water feeding tank in the housing and in fluid communication with the sterilizing chamber; a supply water inlet; a coolant tank in the housing and in fluid communication with the supply water inlet; and an evaporating tank in the housing and having an evaporating tank supply water inlet in fluid communication with an outlet of the coolant tank, and an evaporating tank steam outlet in fluid communication with the distilled water feeding tank.

The sterilizer system may further comprise: a distiller system condenser; a coolant water recirculation fluid circuit between the coolant tank and the distiller system condenser and configured to recirculate supply water between the coolant tank and the distiller system condenser; and a supply water flow circuit from the coolant tank to the evaporating tank and from the evaporating tank back through the coolant tank and to the distilled water feeding tank.

The sterilizer system may further comprise a closed steam conduit extending through the coolant tank, wherein the closed steam conduit is in fluid communication with the evaporating tank steam outlet. The closed steam conduit may further comprise a coil positioned in the coolant tank and configured for heat transfer between steam inside of the coil and supply water inside of the coolant tank.

The sterilizer system may further comprise a recirculating tank in fluid communication with an exhaust outlet of the sterilizing chamber. The sterilizer system may further comprise a sterilizer condenser receiving exhaust from the exhaust outlet and configured to convert the exhaust to water, and a recirculating tank fluid supply line between the sterilizer condenser and the recirculating tank. The recirculating tank may further comprise a recirculating tank outlet in fluid communication with an evaporating tank inlet on the evaporating tank.

The sterilizer system may further comprise a controller configured to initiate evaporating cycles in the evaporating tank based on a detected volume of distilled water in the distilled water feed tank. The controller may be further configured to cause the sterilization system to automatically flush residual dissolved solids after each evaporating cycle.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is a schematic view of a tabletop steam sterilizer system in accordance with certain aspects of an embodiment of the invention.

FIG. 2 is a schematic view of an electronic control system for the tabletop steam sterilizer system of FIG. 1.

FIG. 3 is a key legend depicting the elements of the tabletop steam sterilizer system of FIGS. 1 and 2.

FIG. 4 is a perspective view of a tabletop steam sterilizer system in accordance with certain aspects of an embodiment of the invention.

### DETAILED DESCRIPTION OF THE PREFERRED EMBODIMENTS

The invention may be understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

Unless otherwise indicated, all dimensions shown in the attached drawings are exemplary only and should not be construed as limiting the scope of the invention to those specific dimensions.

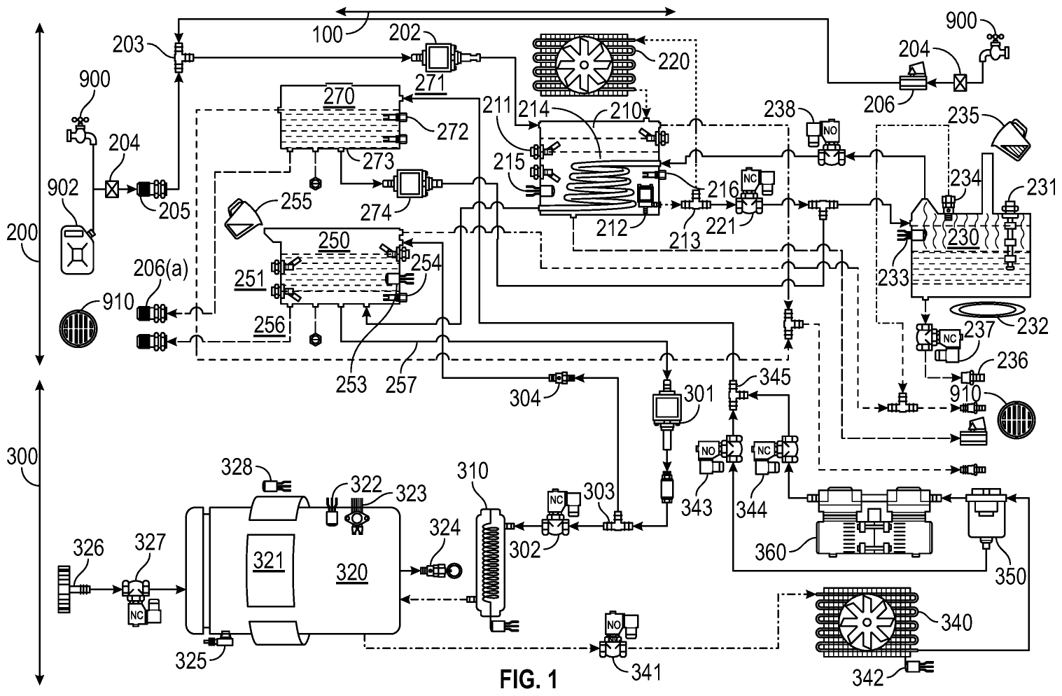

In accordance with certain aspects of an embodiment of the invention and with particular reference to the schematic system view of FIG. 1, a tabletop steam sterilizer system 100 is provided that operates independent of an external supply of distilled water, such that system 100 may be operated from more common water sources, such as water from a municipal water supply from a standard water faucet 900 at the facility at which system 100 is to be used. To achieve such operation, the system includes an onboard, autonomously controlled water distiller system (shown generally at 200). As discussed in greater detail below, the water distiller system 200 is configured to supply distilled water as output to a sterilizer (shown generally at 300) including steam generator 310 that feeds at least one sterilizing chamber 320 of the system. Further, water distiller system 200 may be further configured to receive recirculated distilled water from the sterilizer 300. Systems configured in accordance with at least certain aspects of the invention may convert supply water, such as municipal water from a standard tap, having total dissolved solids (“TDS”) of not more than 500 ppm to distilled water at not more than 9 ppm, and thus may be operated independently from the need for any external supply of distilled water.

The individual components of tabletop steam sterilizer system 100 including both water distiller system 200 and sterilizer 300 will now be described in detail with continuing reference to FIG. 1.

### Water Distiller System (200)

In operation, feed pump 202, such as a vibration pump, draws water on demand from, for example, a standard water faucet 900 connected to a municipal water supply through an external sediment filter 204, such as a readily commercially available 10-micron particulate filter, and delivers that water to a cooling tank 210. Optionally, multiple sources of municipal water may supply feed pump 202 through a 3-way plumbing connector 203. The 3-way plumbing connector 203 may optionally receive municipal feed water through, for example, a quick disconnect valve 205 oriented to front, through a ball valve 206 oriented to rear, or both. Further, in the event that the municipal water supply should become unavailable, an alternative supply (such as a portable container 902 of water having not more than 9 ppm TDS) may likewise be used. Those skilled in the art will recognize that one or more of the components between feed pump 202 and water supply 900 may be positioned either within or outside of tabletop steam sterilizer system 100 without departing from the spirit and scope of the invention.

Float level sensors 211 may detect the level of supply water in cooling tank 210 (which supply water is used as the coolant in cooling tank 210). Based on the detected level of supply water in the cooling tank 210, the system controller may cause feed pump 202 to continue feeding water to cooling tank 210 on demand to maintain the cooling at the desired level. When a pre-designated level of water is reached in cooling tank 210, float level sensor 211 signals a system controller (not shown) to switch on a submersible circulation pump 212 that is immersed inside of the cooling tank 210. The supply water, serving as the coolant in cooling tank 210, is pumped by submersible circulation pump 212 to, for example, a 3-way plumbing connection 213, having a first fluid circuit that feeds a forced-air condenser 220, which continuously cools the supply water and returns it to cooling tank 210 after dispelling the accrued latent heat of steam condensation.

When sterilizer 300 requires distilled water for a steam sterilizing cycle, an electric valve 221, such as a normally closed (“NC”) solenoid valve, directs supply water from the cooling tank 210 through a second fluid circuit of 3-way plumbing connection 213 into the evaporating tank 230. A level sensor 231, such as a vertical float sensor, in evaporating tank 230 senses when the evaporating tank 230 is full and serves as an interlock to preferably switch off the feed pump 202 and to switch on the evaporating heater 232 to start the evaporating process. More particularly, float level sensor 231 is preferably configured to monitor the level of water inside evaporating tank 230 and communicates with the system controller to cause additional water to be drawn from cooling tank 210 when necessary to generate additional steam for condensation into distilled water in an evaporating cycle. In the evaporating tank, electric resistance heating from heater 232 raises the temperature of water to its boiling point, vaporizing approximately 90% of the water and leaving behind all undissolved sediments and dissolved solids in the 10% residual water. Evaporating tank 230 also preferably includes a temperature sensor 233 that communicates with the system controller to ensure that the water inside of evaporating tank 230 is being properly heated by evaporating heater 232 to generate sufficient steam for delivery to coil 214 inside of cooling tank 210. A steam pressure auto vent valve 234 is also provided to ensure that the pressure inside of evaporating tank 230 does not exceed an intended level, with such vent valve 234 venting to an external drain 910.

After each evaporating cycle, the residual water is preferably flushed from evaporating tank 230. A drain 236 is likewise provided in fluid communication with evaporating tank 230 for flushing after preferably each evaporating cycle, preferably with an NC solenoid valve 237 emptying the remaining water and undissolved solids from evaporating tank 230 and directing them to external drain 910. As noted above, evaporating tank 230 is programmed to shut off upon vaporizing approximately 90% of the water in the evaporating tank 230, leaving behind all precipitated sediments and dissolved solids in the residual 10% water, which is flushed preferably after each evaporating cycle. However, with every evaporation cycle, dissolved solids precipitate and adhere to the walls of the evaporating tank 230 as scales. The evaporating tank is built of a 230 SAE 30316 Stainless Steel to AMS 5524L 18Cr-13Ni-2.5Mo. This is a molybdenum alloyed austenitic stainless steel having better resistance to corrosion and pitting from marine environments, and high creep strength at elevated temperatures.

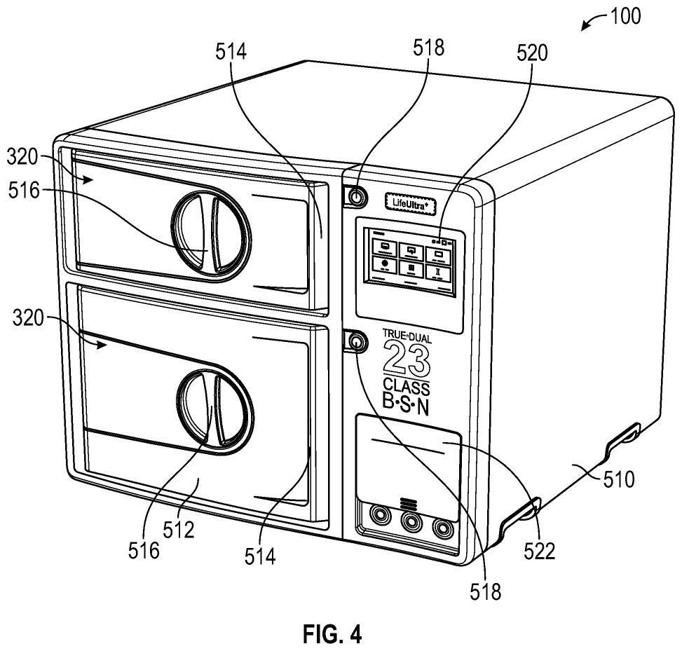

The composition of scales is predominantly calcium, magnesium, and silica. To inhibit the scales from binding, the inner wall of the evaporating tank 230 is preferably electro-polished to a high level of surface smoothness corresponding to IS N5 grade (0.4 microns RA). To control scale buildup inside of evaporating tank 230, tabletop steam sterilizer system 100 preferably automatically prompts the user at regular intervals to undertake a descaling cycle using a prescribed liquid descaling agent, such as Urnex Dezcal. To carry out the process, the user feeds 1 unit of the descaling agent into evaporating tank 230, through inlet 235 and the system controller feeds 10 units of water in proportion to fill the evaporating tank 230. The user enables the descaling cycle from a user-interface display (UI) 520 (FIG. 4), and then fills the descaling agent into the evaporating tank 230 until the UI informs them that the required level is achieved. Then the system controller fills evaporating tank 230 by activating submersible circulation pump 212 until the level of water reaches the overflow level in the evaporating tank 230. Then the evaporating heater 232 is enabled and maintains the solvent at 80° C. for 30 minutes, flushes the solvent, and then rinses the interior of evaporating tank 230 with supply water. During such descaling cycle, the controller likewise closes fluid communication from evaporating tank 230 to cooling tank 210 so that no descaling fumes are carried to the feeding tank 250.

As steam from the evaporating tank 230, devoid of any dissolved solids, rises by natural convection into thin-gauge stainless steel coil 214 positioned inside cooling tank 210, it preferably passes through a normally open (“NO”) solenoid valve 238, which may be closed during a descaling operation in evaporating tank 230. In coil 214, steam transfers latent heat of condensation to the coolant supply water inside of cooling tank 210, and in the process condenses and distils as water. Cooling tank 210 employs water cooling, as its isobaric specific heat is over 4 times better than air cooling, and as noted above, the municipal facility water itself serves as the coolant in the cooling tank 210 prior to it being fed into evaporating tank 230. Further, coil 214 directs now condensed, distilled water to feeding tank 250, from where steam generator 310 of sterilizer 300 draws its required quantities on demand. To ensure that the coolant water inside cooling tank 210 maintains a sufficiently low temperature to effectively condense steam in coil 214, a temperature sensor 215 is provided to constantly monitor the temperature of such coolant water. Should the temperature of coolant water rise above a defined threshold, the system controller may then turn off the evaporating heater 232 until the temperature falls below the defined threshold. Further, cooling tank 210 preferably includes a TDS sensor 216 to enable system controller to monitor TDS of supply water to ensure that water that is directed to evaporating tank 230 does not bear TDS greater than 500 ppm.

Feeding tank 250 preferably includes one or more level sensors 251, such as horizontal float level sensors, that are configured to detect and confirm a level of distilled water in feeding tank 250 that is ready for supply to sterilizer 300. Water level sensors 251 preferably communicate with the system controller to initiate evaporating cycles in evaporating tank 230 as described above whenever necessary to ensure that an intended store of distilled water is constantly maintained in feeding tank 250 for supply to sterilizer system 300. Feeding tank 250 includes an outlet 257 that feeds distilled water from feeding tank 250 to another feed pump 301 of sterilizer 300, which is configured to direct distilled water to steam generator 310 when necessary for generating saturated steam needed to perform a sterilizing cycle in the sterilizing chamber 320 as discussed in greater detail below.

Feeding tank 250 preferably includes a TDS sensor 254 to ensure that distilled water delivered in it has a TDS that does not exceed an intended level for a sterilizing cycle, such as a TDS of not more than 9 ppm. TDS is based on measurement of electrical conductivity in water which is influenced by water temperature, so the feeding tank 250 also preferably includes a temperature sensor 253. TDS readings from the feeding tank 250 are correlated with temperature readings and proportionally compensated to get real values. Further, feeding tank 250 may further have an inlet 255 for receiving distilled water during a manual fill operation, which may be desirable in instances in which both the supply of distilled water in feeding tank 250 is low and a component of water distiller 200 has become temporarily unavailable, but a sterilizing cycle is desired to be carried out. Furthermore, feeding tank 250 may include a drain port 256 that may be used to empty feeding tank 250 into an external drain 910 when desired, such as for carrying out routine maintenance.

Next, recirculating tank 270 may also be provided in water distiller system 200 for reusing distilled water from sterilizer 300 after a sterilizing cycle has been carried out. Residual water after a sterilizing cycle in the sterilizing chamber 320 of the sterilizer 300 may be directed to recirculating tank 270 through an inlet 271 after it has condensed from steam. Optionally, a filter (not shown) may also be provided to filter water received by recirculating tank 270 from sterilizer 300 to ensure that any residual dead pathogens are isolated. Distilled water returning from a sterilizing cycle maintains a TDS under 9 ppm, and more preferably a TDS under 2 ppm (corresponding to electrical conductivity of under 3 μS/cm at 20° C.). Due to physical constraints, recirculating tank 270 preferably includes a level sensor 272, such as an electrical conductivity level sensor configured to detect when the recirculating tank 270 is full. When the recirculating tank 270 is full the system controller may operate pump 274 to direct recycled, distilled water through an outlet 273 in recirculating tank 270 to evaporating tank 230 and so reintroduce the recycled, distilled water back into the system. Further, additional quick disconnect valves 206(a) may be in fluid communication with outlets from each of feeding tank 250 and recirculating tank 270 to allow draining of each of those tanks into a standard external drain 910.

### Sterilizer (300)

Sterilizer 300 receives distilled water from water distiller system 200 by way of feed pump 301 drawing distilled water from feeding tank 250 when required for a sterilizing cycle. Water from feed pump 301 is directed to steam generator 310 preferably through NC solenoid valve 302, which may be opened by the system controller on demand when a sterilizing cycle is underway. Preferably, a 3-way plumbing connector 303 is provided upstream from NC solenoid valve 302, providing a first flow path from feed pump 301 to NC solenoid valve 302, and a second flow path from feed pump 301 back to feeding tank 250, optionally through a water pressure auto vent valve 304, so as to avoid having excess water or water pressure being directed through NC solenoid valve 302 and to steam generator 310.

From steam generator 310, steam is supplied to sterilizing chamber 320 during a sterilizing cycle. Sterilizing chamber 320 includes a chamber heater 321 with heater temperature sensor 328, in addition to a chamber temperature sensor 322 and chamber pressure sensor 323 that are each in communication with the system controller for monitoring sterilizing temperature and pressure values during a sterilizing cycle. Sterilizing chamber 320 also preferably includes a resettable pressure relief valve 324 configured to release excess pressure from sterilizing chamber 320 if and as necessary, and a pneumatically operated door safety lock 325. Sterilizing chamber may receive an air intake for sterilizing cycles through a bacteriological filter 326 and a NC solenoid valve 327 that may be operated by the system controller to open during a sterilizing cycle.

In use, when load is placed inside the sterilizing chamber 320, a program cycle is initiated and the chamber preheats to a set sterilizing temperature. Next, air is removed from the chamber either by gravity or mediated by vacuum pump 360 to −0.8 bar vacuum. To achieve sterility, saturated steam is introduced into sterilizing chamber 320 until sterilizing temperature and pressure are attained (e.g., 121° C. at 1.1 bar or 134° C. at 2.1 bar) and maintained for a set duration (e.g., 4 to 20 min). This kills all bacteria, fungi, viruses, and prions. The saturated steam is then exhausted out of chamber 320 while atmospheric air is drawn in through bacteriological filter 326. The load is dried, again aided by the vacuum pump 360 (e.g., for 5 to 20 min) and then taken out of the chamber once temperature and pressure settle to safe levels.

Following a sterilizing cycle, exhaust steam from sterilizer 300 is then returned to distiller system 200. Once the pressure in the sterilizing chamber 320 reaches a pressure that is equal to atmosphere, NO valve 341 is opened so steam from sterilizing chamber 320 may be exhausted by action of a pump 360, such as a vacuum pump. The exhausted steam is directed through a forced-air condenser 340, to condense the exhausted steam and return such condensed water and steam mixture to recirculating tank 270. Forced-air condenser 340 may also be equipped with a temperature sensor 342, which may communicate with the system controller to allow control of forced-air condenser 340 to ensure adequate condensing of the exhausted steam back into distilled water.

As such condensed water and steam mixture exits forced-air condenser 340 it preferably is drawn through air-water separator 350. Air-water separator 350 separates the air from the water by gravity where the air is drawn through the vacuum pump 360 and water drops down by gravity. The air-water separator 350 is upstream of the vacuum pump 360 because the vacuum pump 360 is configured to exhaust only residual steam and air from the sterilizing chamber 320. Air and condensed steam from the vacuum pump 360 are regulated by NC solenoid valve 344 and water output from air-water separator 350 is regulated by NO solenoid valve 343 as they both combine through 3-way plumbing connection 345 and are directed into recirculating tank 270 that may supply evaporating tank 230 with additional condensed water and avoid wastage.

### Electrical Schematic

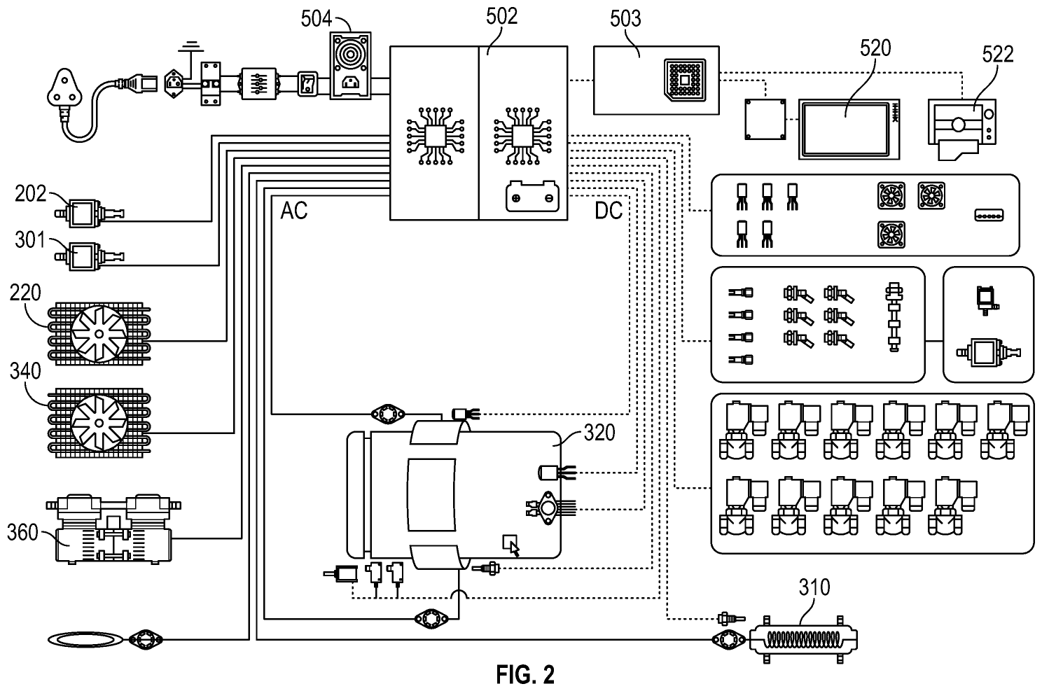

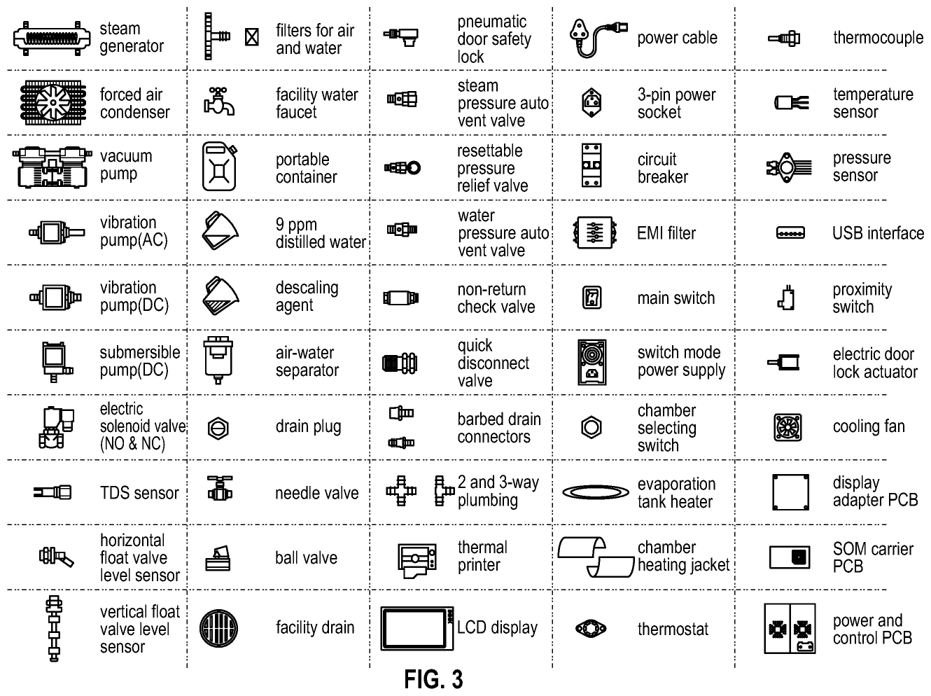

As shown in the electrical schematic of FIG. 2 (and with reference to the system component legend of FIG. 3) for tabletop steam sterilizer system 100, the various heat exchangers that provide heating and cooling, along with pumps, electro-mechanical valves, pressure vent valves, and non-return valves that control the rate and instances of water and steam flow within and between distiller system 200 and sterilizer 300 as discussed above are preferably operated by an electronic controller (shown generally at 502) and a system-on-module (SOM) board 503 having software instructions stored thereon with inputs from the various level sensors, TDS sensors, temperature sensors, thermocouples, thermostats, and pressure sensors discussed above. A PID control-feedback loop system is implemented on the heaters for optimum control and to prevent temperature from overshooting safe thresholds. A smart switching sequence is implemented between the heaters: chamber heater 321, steam generator 310, and evaporating heater 232 which switches power from power supply 504 among themselves in a specific time interval such that the overall power of the machine does not cross 2 kVA.

### Desktop Unit

In accordance with certain aspects of an embodiment of the invention, and with particular reference to FIG. 4, tabletop steam sterilizer system 100 configured as above is contained within a compact, desktop or tabletop cabinet housing 510, providing a significantly reduced profile that conserves space in comparison to traditional sterilization systems, and thus may be suitable for deployment in smaller medical and dental practices that tend to have limited space available for such equipment. Tabletop steam sterilizer system 100 includes at least one sterilizing chamber 320 configured as above, and in certain exemplary configurations may include more than one sterilization chamber 320. The interior of each such sterilization chamber 320 may be accessed via a door 512. To open a sterilization chamber 520, a user may grasp handle 514 and pull the door away from the sterilizing chamber 320, pivoting the door about a hinge at the opposite edge from handle 514. Preferably, cabinet housing 510 is configured to enable door 512 to slide into cabinet housing 510 after it has been opened, thus positioning the door inside of cabinet housing 510 and next to a side wall of cabinet 510 while the user places elements that are to be sterilized into the sterilization chamber 320. A door lock 516 is provided on the front of the door that may be manually operated to seal a closed door 512 against its respective sterilization chamber 320 when in use. In those configurations in which more than one sterilizing chamber is provided, a chamber selection switch 518 may be provided that enables a user to select which of the multiple sterilizing chambers 320 are to currently be used. An LDC display 520, such as a touch-responsive display, is preferably provided on the front of cabinet 510 to enable a user to engage the operations of tabletop steam sterilizer system 100 discussed herein. With reference again to the schematic system view of FIG. 1, and in accordance with a particular aspect of an exemplary embodiment, cabinet housing 510 preferably houses all components of tabletop steam sterilizer system 100 in the single cabinet housing 510, including a water distiller system 200 and sterilizer 300. Likewise, cabinet housing 510 holds all other elements of tabletop steam sterilizer system 100, which those of ordinary skill in the art will recognize may include the steam generator, a processor, a power system, control firmware, transformer, battery, printer, USB interface, WiFi communication module, heat jackets, thermostats, condensers, vacuum pump, water pumps, solenoids, gauges, meters, temperature and pressure sensors, safety locks, switches, and the like, many of which elements are of typical configuration and thus known to those of ordinary skill in the art and may be variously adapted to tabletop steam sterilization system 100 as described herein, such that the specific configurations of those individual elements is not further described herein.

Systems configured in accordance with at least certain aspects of the invention may result in one or more advantages over previously known sterilizing systems. By way of non-limiting example, the process employed by the system 100 of flushing residual dissolved solids after each evaporating cycle, and with descaling cycles at regular intervals, allows the distiller system 200 of tabletop steam sterilizer system 100 to process 500 ppm input water to 9 ppm distilled water for use in sterilizer 300, thus operating independently of an external supply of distilled water. Further, water distiller system 200 employs water cooling, which is over 4 times more efficient than air cooling. In this configuration, no separate coolant is used, but rather the municipal facility supply water itself serves as the coolant in cooling tank 210 prior to being fed to evaporating tank 230, thereby saving on coolant, reducing the heating load in evaporating tank 230, and improving overall energy efficiency. Furthermore, the system controller is configured to provide operation of all three sterilizing cycle classes—i.e., B, S, & N.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

1. A sterilizer system, comprising:
a housing;
at least one sterilizing chamber in said housing; and
a water distiller system in said housing and in fluid communication with said at least one sterilizing chamber, wherein said water distiller system has a municipal water supply inlet configured to receive water from a municipal water supply, and a distilled water outlet in fluid communication with said at least one sterilizing chamber;
wherein said water distiller system is configured to convert municipal water into distilled water for supply to said at least one sterilizing chamber.

2. The sterilizer system of claim 1, further comprising a sterilizer condenser in said housing having an inlet in fluid communication with an exhaust from said at least one sterilizing chamber and an outlet in fluid communication with said water distiller system.

3. The sterilizer system of claim 1, said water distiller system further comprising:
a cooling tank in fluid communication with said municipal water supply inlet;
a distiller system condenser;
a coolant water recirculation fluid circuit between said coolant tank and said distiller system condenser and configured to recirculate water between said coolant tank and said distiller system condenser; and
a supply water flow circuit from said coolant tank to an evaporating tank and from said evaporating tank back through said coolant tank and to a distilled water feeding tank, wherein said distilled water feeding tank is in fluid communication with said distilled water outlet.

4. The sterilizer system of claim 3, further comprising a closed steam conduit extending through said coolant tank, wherein said closed steam conduit is in fluid communication with a steam outlet on said evaporating tank and a distilled water inlet on said distilled water feeding tank.

5. The sterilizer system of claim 4, said closed steam conduit further comprising a coil positioned in said coolant tank and configured for heat transfer between steam inside of said coil and municipal water inside of said coolant tank.

6. The sterilizer system of claim 3, said water distiller system further comprising a recirculating tank in fluid communication with an exhaust outlet of said at least one sterilizing chamber.

7. The sterilizer system of claim 6, further comprising a sterilizer condenser receiving exhaust from said exhaust outlet and configured to convert said exhaust to water, and a recirculating tank fluid supply line between said sterilizer condenser and said recirculating tank.

8. The sterilizer system of claim 6, said recirculating tank further comprising a recirculating tank outlet in fluid communication with an evaporating tank inlet on said evaporating tank.

9. The sterilizer system of claim 3, further comprising a controller configured to initiate evaporating cycles in said evaporating tank based on a detected volume of distilled water in said distilled water feed tank.

10. The sterilizer system of claim 9, wherein said controller is further configured to cause said sterilizer system to automatically flush residual dissolved solids after each evaporating cycle.

11. A sterilizer system, comprising:
a housing;
a sterilizing chamber in said housing;
a distilled water feeding tank in said housing and in fluid communication with said sterilizing chamber;
a supply water inlet;
a coolant tank in said housing and in fluid communication with said supply water inlet; and
an evaporating tank in said housing and having an evaporating tank supply water inlet in fluid communication with an outlet of said coolant tank, and an evaporating tank steam outlet in fluid communication with said distilled water feeding tank.

12. The sterilizer system of claim 11, further comprising:
a distiller system condenser;
a coolant water recirculation fluid circuit between said coolant tank and said distiller system condenser and configured to recirculate supply water between said coolant tank and said distiller system condenser; and
a supply water flow circuit from said coolant tank to said evaporating tank and from said evaporating tank back through said coolant tank and to said distilled water feeding tank.

13. The sterilizer system of claim 12, further comprising a closed steam conduit extending through said coolant tank, wherein said closed steam conduit is in fluid communication with said evaporating tank steam outlet.

14. The sterilizer system of claim 13, said closed steam conduit further comprising a coil positioned in said coolant tank and configured for heat transfer between steam inside of said coil and supply water inside of said coolant tank.

15. The sterilizer system of claim 11, further comprising a recirculating tank in fluid communication with an exhaust outlet of said sterilizing chamber.

16. The sterilizer system of claim 15, further comprising a sterilizer condenser receiving exhaust from said exhaust outlet and configured to convert said exhaust to water, and a recirculating tank fluid supply line between said sterilizer condenser and said recirculating tank.

17. The sterilizer system of claim 15, said recirculating tank further comprising a recirculating tank outlet in fluid communication with an evaporating tank inlet on said evaporating tank.

18. The sterilizer system of claim 11, further comprising a controller configured to initiate evaporating cycles in said evaporating tank based on a detected volume of distilled water in said distilled water feed tank.

19. The sterilizer system of claim 18, wherein said controller is further configured to cause said sterilization system to automatically flush residual dissolved solids after each evaporating cycle.
